(19) United States
(12) Patent Application Publication
(10) Pub. No.: US 2021/0315500 A1
(43) Pub. Date: Oct. 14, 2021

(54) SYSTEMS AND METHODS FOR DETERMINING WELLNESS USING A MOBILE APPLICATION

(71) Applicant: döTERRA International LLC, Pleasant Grove, UT (US)

(72) Inventors: Dell Brown, Pleasant Grove, UT (US); John Dye, Pleasant Grove, UT (US); Tate Knutstad, Aurora, UT (US); Henry J. O'Connell, Spanish Fork, UT (US); Seth Winters, Pleasant Grove, UT (US)

(21) Appl. No.: 17/226,392

(22) Filed: Apr. 9, 2021

Related U.S. Application Data
(60) Provisional application No. 63/008,084, filed on Apr. 10, 2020.

(51) Int. Cl. A61B 5/16; A61B 5/00; G16H 10/20

(52) U.S. Cl. CPC A61B 5/165; G16H 10/20; A61B 5/743; A61B 5/749

(57) ABSTRACT

Disclosed are various embodiments of systems and method of determining wellness of a user using a mobile application. In some examples, a system can display a first wellness prompt on a client device for a first wellness assessment. The system can receive a first audio sample from the client device. A first wellness assessment can be generated based at least in part on the first audio sample. Wellness content can be transmitted to the client device. The system can display a second wellness prompt on the client device for a second wellness assessment. The system receive a second audio sample from the client device and generate the second wellness assessment based on the second audio sample. The first wellness assessment and the second wellness assessment can be displayed for the client device.

### CROSS REFERENCE TO RELATED APPLICATIONS

This application claims priority to, and the benefit of, U.S. Ser. No. 63/008,084 filed Apr. 10, 20120 and entitled “SYSTEMS AND METHODS FOR DETERMINING WELLNESS USING A MOBILE APPLICATION,” which is incorporated by reference herein in its entirety.

### BACKGROUND

Individuals can develop feelings of stress from a particular event or from concerns that can cause frustration, anger, or nervousness. In some cases, stress may subside over time. In other cases, individuals may have a difficult time alleviating their stress even after time has passed. In these cases, the wellness or health of an individual can be impacted by prolonged feelings of stress. As such, there is a need for a systematic solution for determining and tracking the wellness of an individual as treatment methods and products are implemented to reduce the level of stress being experienced by the individual.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a drawing of a networked environment according to various embodiments of the present disclosure.

FIGS. 2A-2F illustrate example user interfaces for the client application of FIG. 1, according to one embodiment described herein.

FIGS. 3A-3D illustrate example user interfaces for the client application of FIG. 1, according to one embodiment described herein.

FIG. 4 is a flowchart illustrating one example of baseline workflow functionality implemented as portions of the wellness application executed in a computing environment in the networked environment of FIG. 1 according to various embodiments of the present disclosure.

FIG. 5 is a flowchart illustrating one example of course workflow functionality implemented as portions of the wellness application executed in a computing environment in the networked environment of FIG. 1 according to various embodiments of the present disclosure.

FIG. 6 is a flowchart illustrating one example of journal workflow functionality implemented as portions of the wellness application executed in a computing environment in the networked environment of FIG. 1 according to various embodiments of the present disclosure.

FIG. 7 is a schematic block diagram that provides one example illustration of a computing environment employed in the networked environment of FIG. 1 according to various embodiments of the present disclosure

### DETAILED DESCRIPTION

The embodiments of the present disclosure relate to systems and methods that can determine the wellness or health of an individual and track a progression of whether the wellness of the individual is improving. The embodiments can include interfacing with a speech service for determining various health conditions based on an audio sample of the user's voice. Additionally, the systems and methods can allow the individual to take a bio-feedback questionnaire and can provide the individual's responses to the speech service. The speech service can calculate a baseline score and subsequent wellness scores related to stress, anxiety, quality of sleep, or other health conditions.

The systems and methods can also obtain responses corresponding to affirmations, questions, or other prompts to help the individual track his or her stress-reduction goals. For example, the systems and methods can obtain a first response which is a recording of the individual reading an affirmation (e.g., verbally reading aloud a script or describing what is seen in an image) or another prompt out loud and prior to the individual completing a guided meditation session that is available for streaming from a content library. The systems and methods can obtain a second response after the individual completes the guided meditation session. The systems and methods can provide the first and the second response to the speech service to assess the individual's improvement related to his or her scores for stress, anxiety, quality of sleep, or other health-related conditions. The systems and methods can generate a user interface including score(s) received from the speech service.

In some examples, the systems and methods can allow the individual to assess his or her progress via journaling. The systems and methods can obtain response(s) including an emoji selection, multiple choice value, and/or a recorded response (e.g., a 16 bit depth, mono channel, 44.1 KHz sample rate recording of the individual's speech that is stored in an uncompressed or lossless compressed format). The systems and methods can provide the response(s) to the speech service for the assessment of stress, anxiety, sleep, or other conditions. The systems and methods can request score(s) from the speech service. The systems and methods can generate a user interface including score(s) received from the speech service.

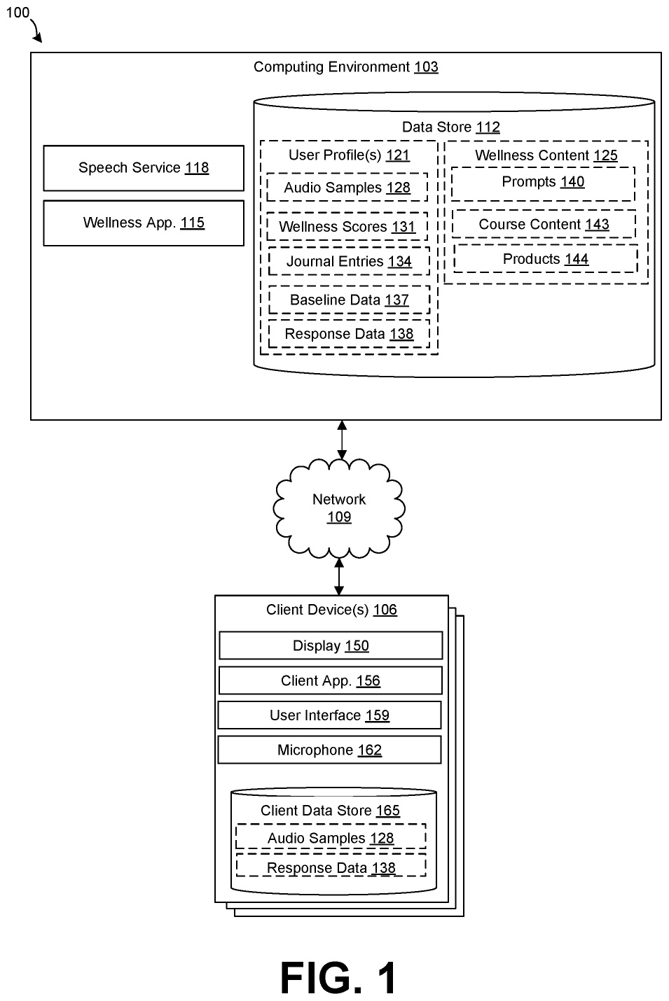

With reference to FIG. 1, shown is a networked environment 100 according to various embodiments. The networked environment 100 includes a computing environment 103, and a client device 106, which are in data communication with each other via a network 109. The network 109 includes, for example, the Internet, intranets, extranets, wide area networks (WANs), local area networks (LANs), wired networks, wireless networks, or other suitable networks, etc., or any combination of two or more such networks. For example, such networks may comprise satellite networks, cable networks, Ethernet networks, and other types of networks.

The computing environment 103 may comprise, for example, a server computer or any other system providing computing capability. Alternatively, the computing environment 103 may employ a plurality of computing devices that may be arranged, for example, in one or more server banks or computer banks or other arrangements. Such computing devices may be located in a single installation or may be distributed among many different geographical locations. For example, the computing environment 103 may include a plurality of computing devices that together may comprise a hosted computing resource, a grid computing resource and/or any other distributed computing arrangement. In some cases, the computing environment 103 may correspond to an elastic computing resource where the allotted capacity of processing, network, storage, or other computing-related resources may vary over time.

Various applications and/or other functionality may be executed in the computing environment 103 according to various embodiments. Also, various data is stored in a data store 112 that is accessible to the computing environment 103. The data store 112 may be representative of a plurality of data stores 112 as can be appreciated. The data stored in the data store 112, for example, is associated with the operation of the various applications and/or functional entities described below.

The components executed on the computing environment 103, for example, the wellness application 115, the speech service 118, and other applications, services, processes, systems, engines, or functionality not discussed in detail herein. The wellness application 115 is executed to communicate wellness content with one or more client devices 106. The speech service 118 is executed to analyze audio samples in order to determine a level of stress, anxiety, wellness, and other health-related conditions. The speech service 118 analyzes different voice biomarkers in the audio sample to determine a level of stress, anxiety, and other health-related conditions represented in the voice of the user. The wellness application can use an application programing interface (API) to transmit data to the speech service 118 and receive data from the speech service 118 in a standardized manner.

The data stored in the data store 112 includes, for example, user profiles 130, wellness content 125, and potentially other data. The user profile 121 can include audio samples 128, wellness scores 131, journal entries 134, baseline data 137, response data 138, and other suitable user profile data. The audio samples 128 can represent one or more audio files that recorded the voice of a user. In some embodiments, the audio samples 128 can be recorded by a microphone of a client device 106. The wellness application 115 can render on the client device 106 a text prompt or an image for capturing the voice of the user for the audio sample 128. For example, the wellness application 115 can instruct the user to verbally recite a text prompt or verbally describe an image displayed on the user interface.

The wellness scores 131 can represent one or more scores that have been generated to reflect a level of stress, anxiety, wellness, quality of sleep, and other health-related conditions. The wellness scores 131 can be generated by the speech service 118 based on analyzing one or more audio samples 128. The wellness scores 131 can also be generated based on information provided to the wellness application 115. For example, the wellness scores 131 can be generated based on answers to questions displayed by the wellness application 115. In some examples, the wellness scores 131 can be generated by the wellness application 115 and/or by an application executed on the client device 106.

The journal entries 134 can represent one or more text or audio entries provided by the client device 106. The text or audio entries can reflect personal thoughts or comments in response to a feedback prompt displayed on a display of the client device 106. The journal entries 134 can be stored in association with a particular day of the week in a calendar. Accordingly, the wellness application 115 can enable a user to review journal entries 134 in order to track their wellness progress.

The baseline data 137 can reflect information related to an initial wellness assessment of a user. The baseline data 137 can include user responses to a questionnaire. The baseline data 137 can also include an audio sample 128 of an initial prompt, in which the user is verbally reciting a text displayed on a user interface or verbally describing an image displayed on the user interface. The baseline data 137 can also include an initial wellness score 131 that has been calculated from the initial response data 138. The response data 138 can include answers to questions, emoji selections, and other suitable data that reflects a user's feelings or concerns relating to wellness.

The wellness content 125 can include prompts 140, course content 143, products 144, and other related wellness content 125. The prompts 140 can represent various user interfaces that are displayed for the wellness application 115. The prompts 140 are generated and transmitted to the client device 106 for display.

The course content 143 can include wellness material that can be streamed to the client device 106. The course content 143 can include various subject matter courses that can be presented to a user to provide instructions for improving wellness of the user. For example, the course content 143 can include a guided meditation course, a course on techniques for reducing daily stress, a course on improving an individual's focus, and other suitable health related courses. In some cases, the course content 143 can be recommended or automatically selected for the user based on wellness scores 131, baseline data 137, user demographic data, and other suitable data.

The products 144 can include recommendations to the user for use of one or more supplements or essential oils to treat the assessed stress, quality of sleep, anxiety, and other health-related conditions. Some non-limiting examples of products 144 include essential oils capsules, essential oils softgel capsules, essential oils in liquid form, and other suitable aromatic essential oils products. The products 144 can also include recommended devices that facilitate the use of the supplements or essential oils. Some non-limiting examples of these devices can include an aroma humidifier, an aroma diffuser, and other suitable aroma devices.

The wellness application 115 can generate product recommendations and product usage recommendation based on data stored in the user profile 121 (e.g., wellness scores 131, baseline data 137, audio samples 128, journal entries 134, etc.) For example, the wellness application 115 can recommend products 144 based on the wellness score 131 being within a range of values for a particular product. For instance, if a wellness score 131 falls within a first range of values, then the wellness application 115 can recommend a first essential oils product. If the wellness score 131 falls within a second range of values, then the wellness application 115 can recommend a second essential oils product. In some examples, the ranges for the wellness scores 131 may be considered as a low range, a medium range, and a high range. The recommendation can be based on the range that all or a significant number (e.g., a threshold) of the wellness scores 131 falls within.

In some embodiments, the wellness application 115 can recommend a product 144 based on a pattern of wellness scores 131 or if the wellness score 131 meets an elevated threshold. The pattern of wellness scores 131 (or if a wellness score meets a threshold) can also factor into the wellness application 115 recommending a particular dosage of a product 144. For example, after the wellness application 115 detects a different pattern of wellness scores 131 or a wellness score 131 fails below the elevated threshold, then the wellness application 115 can change its dosage recommendation (e.g. a change from four drops to two drops of an essential oils product). The wellness application 115 can also recommend how often the dosage should be taken (e.g., take an essential oils product once a day).

The client device 106 is representative of a plurality of client devices 106 that may be coupled to the network 109. The client device 106 may comprise, for example, a processor-based system such as a computer system. Such a computer system may be embodied in the form of a desktop computer, a laptop computer, personal digital assistants, cellular telephones, smartphones, set-top boxes, music players, web pads, tablet computer systems, game consoles, electronic book readers, or other devices with like capability. The client device 106 may include a display 150. The display 150 may comprise, for example, one or more devices such as liquid crystal display (LCD) displays, gas plasma-based flat panel displays, organic light emitting diode (OLED) displays, electrophoretic ink (E ink) displays, LCD projectors, or other types of display devices, etc.

The client device 106 may be configured to execute various applications such as a client application 156 and/or other applications. The client application 156 may be executed in a client device 106 for example, to access network content served up by the computing environment 103 and/or other servers, thereby rendering a user interface 159 on the display 150. To this end, the client application 156 may comprise, for example, a browser, a dedicated application, etc., and the user interface 159 may comprise a network page, an application screen, etc. The client device 106 may be configured to execute applications beyond the client application 156 such as, for example, email applications, social networking applications, word processors, spreadsheets, and/or other applications. The client device 106 can also include a microphone 162 for recording the voice of the user in order to create audio samples 128.

Various applications and/or other functionality may be executed in the client device 106 according to various embodiments. Also, various data is stored in a client data store 165 that is accessible to the client device 106. The client data store 165 may be representative of a plurality of client data stores 165 as can be appreciated. The data stored in the client data store 165, for example, is associated with the operation of the various applications and/or functional entities described below.

The components executed on the client device 106, for example, include the client application 156, and other applications, services, processes, systems, engines, or functionality not discussed in detail herein. The client application 156 is executed to communicate with the wellness application 115 and execute functionality associated with the wellness application 115. For example, the client application 156 can be executed to display wellness content 125 and record user feedback for analysis. The data stored in the client data store 165 includes, for example, the audio samples 128, response data 138, and potentially other data.

Next, a general description of the operation of the various components of the networked environment 100 is provided. To begin, a user of the client device 106 can enter the appropriate information to set up a user profile 121 as a new user. The user can login to the client application 156 with the user profile 121. The client application 156 can be in data communication with the wellness application 115 in order to display wellness content 125, take wellness assessments (e.g., generate a wellness score 131), store journal entries, and other wellness related functions.

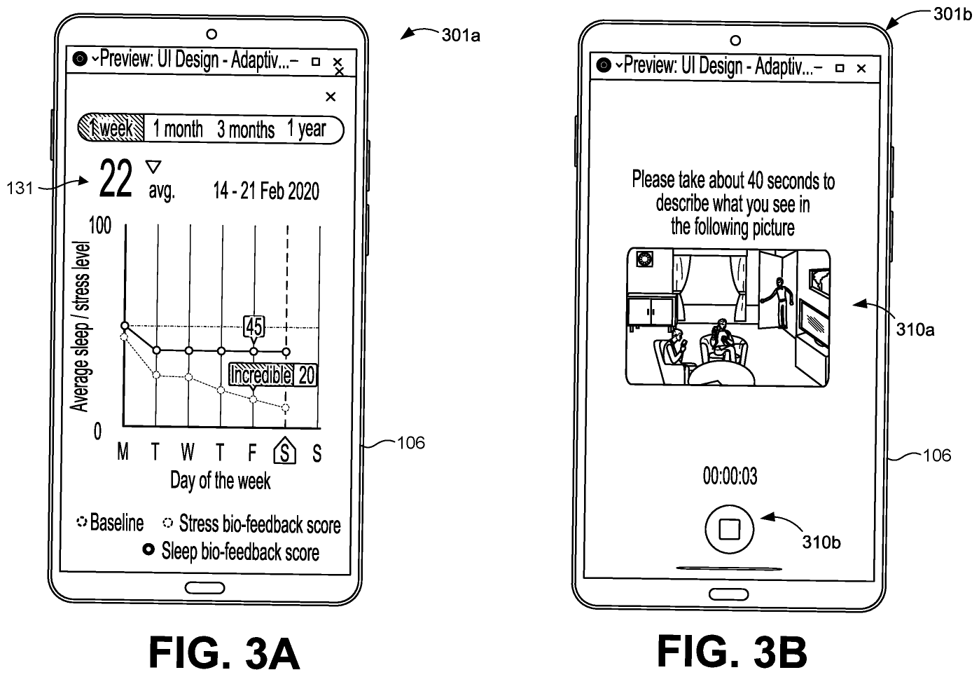

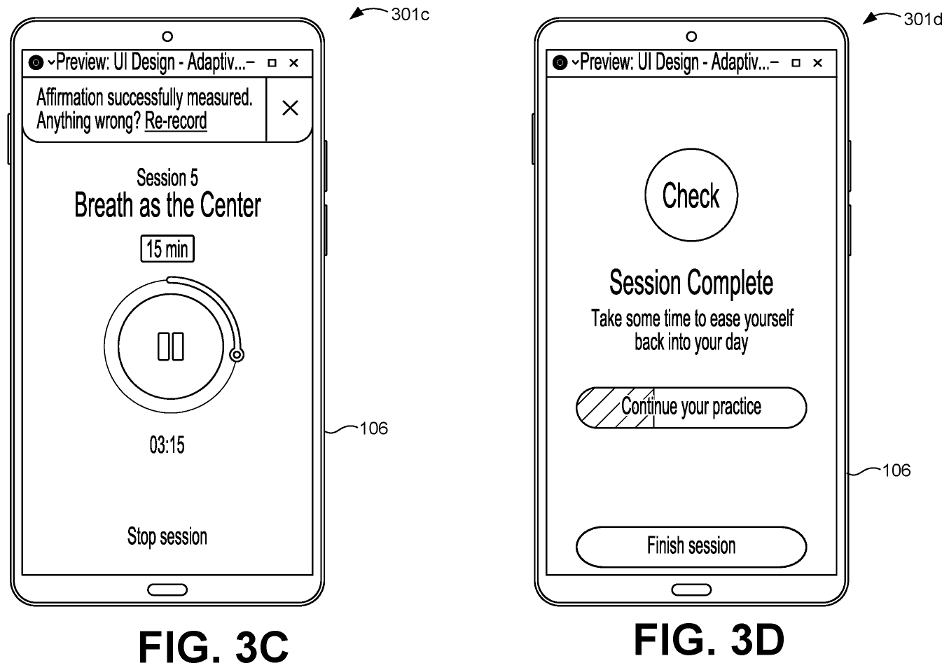

According to one embodiment, as a new user may first perform a baseline workflow (e.g., FIG. 3) in order to collect baseline data 137 for the wellness of the user. The client application 156 can receive and display user interfaces for capturing the baseline data 137. The baseline data 137 can include an initial assessment of stress, quality of sleep, anxiety, and other health-related conditions for the user. The baseline data 137 can later be used in comparison to subsequent wellness assessments in order to track the effectiveness of various stress reducing techniques and products. In some embodiments, the baseline workflow can include obtaining an initial audio sample 128 of the user verbally speaking. The audio sample 128 can be analyzed to provide an initial wellness assessment of the user. The audio sample 128 can be analyzed to identify various biomarkers in the speech to evaluate the level of wellness of the user, such as a level of stress, anxiety, and other health related indicators. Additionally, the baseline workflow can include collecting answers to various health-related questions.

According to one embodiment, the baseline workflow can be used as reference data (e.g., baseline data 137) for subsequent wellness assessments (e.g., wellness scores 131). The reference data can be used to recommend wellness content 125, wellness techniques (e.g., mediation, lifestyle changes, etc.), and/or wellness products (e.g., treatments, oils, etc.).

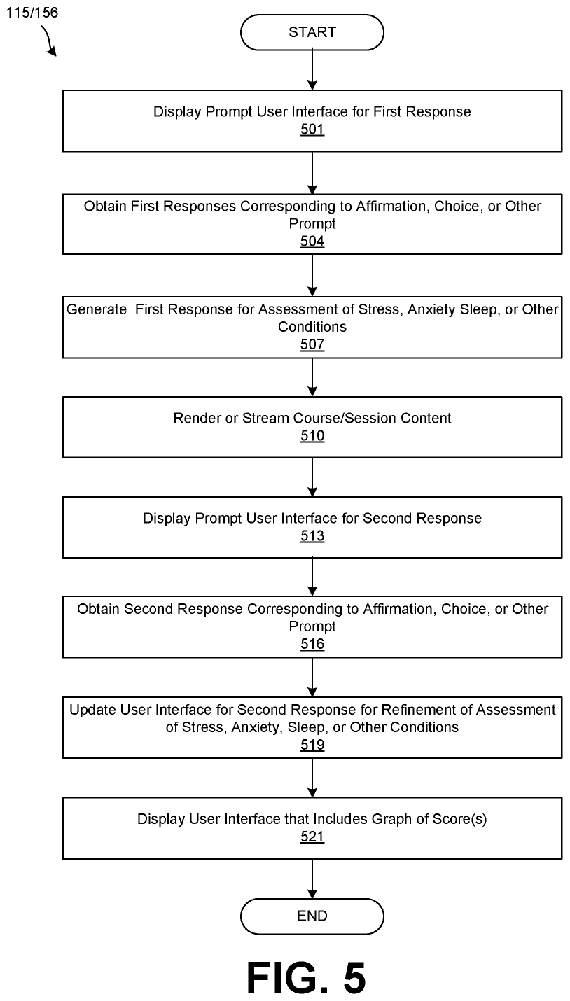

With regard to the wellness content 125, in some embodiments, the wellness application 115 and/or the client application 156 can execute a wellness session workflow (see, e.g., FIG. 5). In some embodiments, the wellness session workflow can include generating a first wellness assessment (a first wellness score 131) for the user to perform. Then, the wellness application 115 can stream or render wellness content 125 to the client device 106. In some examples, the wellness content 125 can be recommended based on the first wellness assessment. The wellness content 125 can include course content 143, such as a course on mediation, breathing techniques, stress management, organization skills, and other suitable courses for reducing stress and improving the overall health of the user. In some embodiments, the course content 143 can be dynamically adjusted based on the user profile 121 (e.g., baseline data 137, wellness scores 131, requested goals of the user, and other suitable factors). As such, the course content 143 can be unique to the user viewing the content.

After the course content 143 has been viewed, the wellness application 115 can generate a second wellness assessment for the user to take. The second wellness assessment can be taken to track the effectiveness of the wellness content 125 that was provided. The wellness application 115 can generate statistics for the impact that the course content 143 had on the user. In some cases, the user may see greater improvement for certain types of course content 143. In other examples, the statistics can be used to recommend a particular course content 143 to a population of subsequent users. For instance, the baseline data 137 and/or the first wellness assessment (e.g., wellness score 131) can be used to recommend the particular course content 143 that has been successful in the past with other users with similar baseline data 137 and/or first wellness assessments. Further, the baseline data 137 can include demographic data that can be factored into the recommendation of the course content 143. The demographic data can include age, height, weight, experience with meditation (e.g. guided or unguided), and other suitable demographic data,

In other examples, the wellness application 115 can recommend wellness products for reducing stress and improving the overall health of the user. For example, the wellness application 115 can recommend products 144 (e.g., aromatic essential oils, aromatic dehumidifier, etc.) for consumption in a beverage, for application to the skin, to diffuse as an aroma, and other suitable applications. The wellness products 144 can be recommended based on the baseline data 137, the wellness scores 131, and goals set for the user (e.g., to improve quality of sleep, reduce stress, improve focus, improve rest, etc.).

For example, the product can be a doTERRA Adaptiv™ capsule. This product contains a blend of CPTG® essential oils Lavender, Coriander, Wild Orange, and Fennel along with Sceletium, GABA, and Ahiflower®. The provided directions recommend 1 capsule per day for mood-boosting and tension-reducing effects. The wellness application 115 can in some embodiments recommend 0 to 4 capsules per day based on the baseline data 137, the wellness scores 131, and goals set for the user.

The product can also be a doTERRA Adaptiv™ oil, which contains a blend of Wild Orange, Lavender, Copaiba, Spearmint, Magnolia, Rosemary, Neroli, and Sweetgum. The provided directions recommend either diffusing three to four drops in a diffuser, or topically applying one to two drops on the skin, without any instructions on the number of times or duration. The wellness application 115 can in some embodiments recommend diffusing or topically applying the oil 0 to 3 times per day based on the baseline data 137, the wellness scores 131, and goals set for the user. For example, as the wellness score improves, the diffusion or topical application could decrease from 3 to 2 to 1 to 0 times per day. Topical application includes application to the wrists, back of neck, front of neck, or temples.

The product can also be a doTERRA Adaptiv™ Touch, which is a blend of Wild Orange, Lavender, Copaiba, Spearmint, Magnolia, Rosemary, Neroli, and Sweetgum in a base of Fractionated Coconut Oil. The provided directions recommend topical or aromatic uses as desired, but no specific instruction on number of times or duration. The wellness application 115 can in some embodiments recommend topical or aromatic application 0 to 3 times per day based on the baseline data 137, the wellness scores 131, and goals set for the user. For example, as the wellness score improves, the topical or aromatic application could decrease from 3 to 2 to 1 to 0 times per day. Topical application includes application to the wrists, back of neck, front of neck, or temples.

The product can also be a doTERRA Serenity® Restful Complex Softgel, which combines the relaxing benefits of Lavender essential oil and L-Theanine along with lemon balm, passionflower, and chamomile to gently promote relaxation and sleep. The provided directions recommend 1 to 2 capsules per day before bed. The wellness application 115 can in some embodiments recommend 0 to 3 times per day before bed based on the baseline data 137, the wellness scores 131, and goals set for the user. For example, as the wellness score improves, the dosage could decrease from 3 to 2 to 1 to 0 capsules per day before bed.

The product can also be a doTERRA Serenity® oil, which contains a blend of Lavender Flower, Cedarwood, Ho Wood Leaf, Ylang Ylang Flower, Marjoram Leaf, Roman Chamomile Flower, Vetiver Root, Vanilla Bean Absolute, and Hawaiian Sandalwood. The provided directions recommend either diffusing three to four drops in a diffuser, or topically applying one to two drops on the skin, without any instructions on the number of times or duration. The wellness application 115 can in some embodiments recommend diffusing or topically applying the oil 0 to 3 times per day based on the baseline data 137, the wellness scores 131, and goals set for the user. For example, as the wellness score improves, the diffusion or topical application could decrease from 3 to 2 to 1 to 0 times per day. Topical application includes application to the wrists, back of neck, front of neck, or temples.

The product can also be a lavender oil, such as the doTERRA Lavender Oil (Lavandula angustifolia). The provided directions recommend diluting one drop in 4 fl oz of liquid for internal use without any instructions on the number of times or duration. The wellness application 115 can in some embodiments recommend taking this dilution internally 0 to 3 times per day or one time before bed based on the baseline data 137, the wellness scores 131, and goals set for the user. For example, as the wellness score improves, the dosage could decrease from 3 to 2 to 1 to 0 times per day.

The first and second wellness assessments can include a user answering a questionnaire with health related questions. The first wellness assessment can include a prompt with instructions for the user to verbally recite a portion of text, in which the voice of the user is recorded as an audio sample 128. In other examples, the user can be instructed to verbally describe an image displayed on the user interface 159 of the client device 106. In either example, the voice of the user can be recorded as an audio sample 128 by the microphone 162 of the client device 106. The audio sample 128 can be transmitted to the wellness application 115 or the speech service 118 for processing, in which a wellness score 131 can be generated based on the audio sample 128. For example, the wellness application 115 can transmit the audio sample 128 to the speech service 118 for processing. The speech service 118 can generate and transmit a wellness score 131 to the wellness application 115. The wellness score 131 can be a numerical value in a range of possible numerical values. As such, the wellness application 115 can track the wellness scores 131 in order to evaluate the progression of the user towards their set goals.

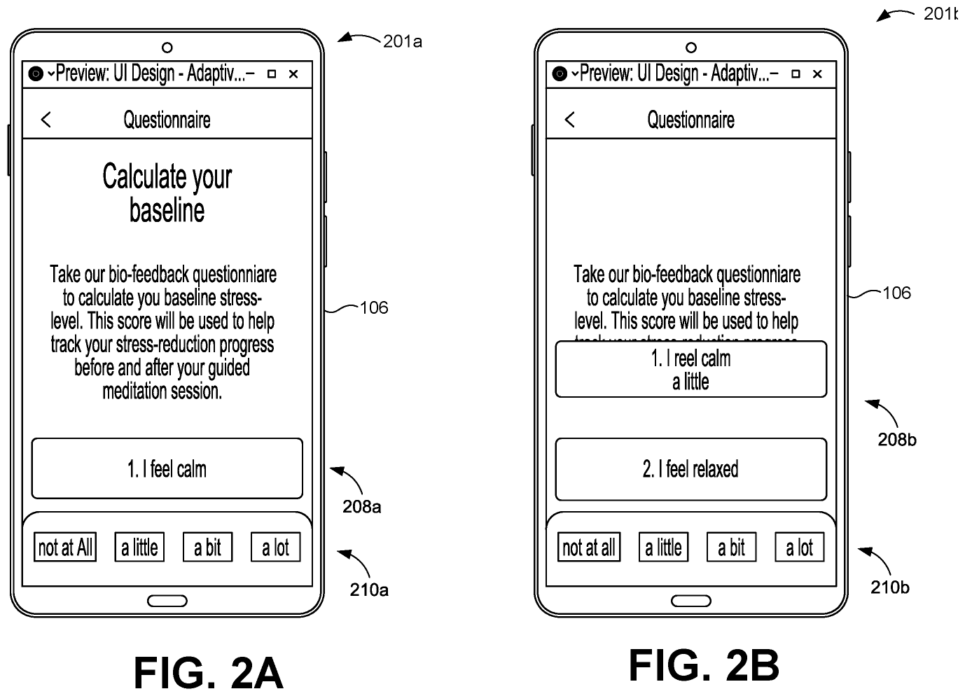

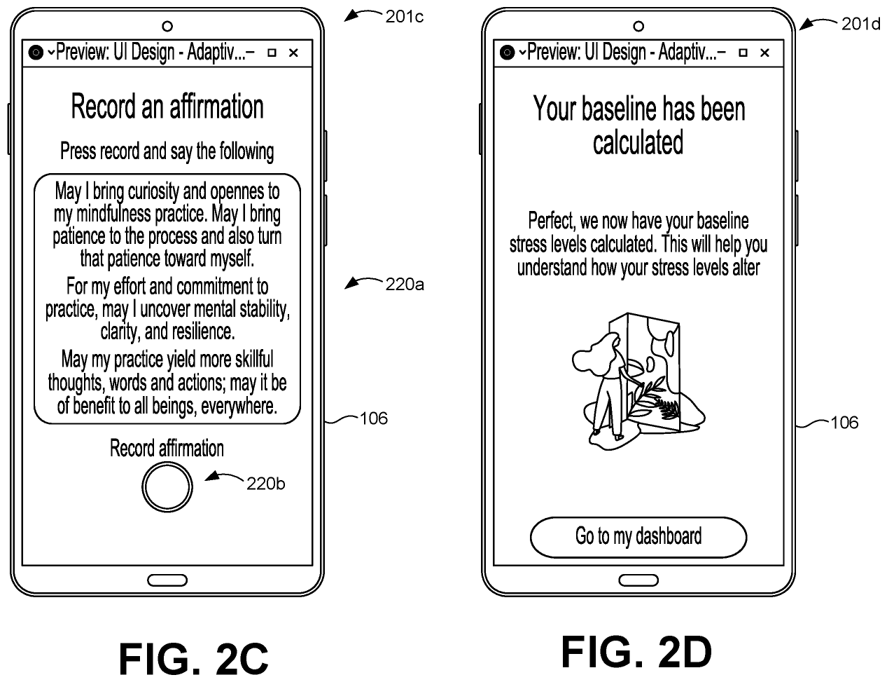

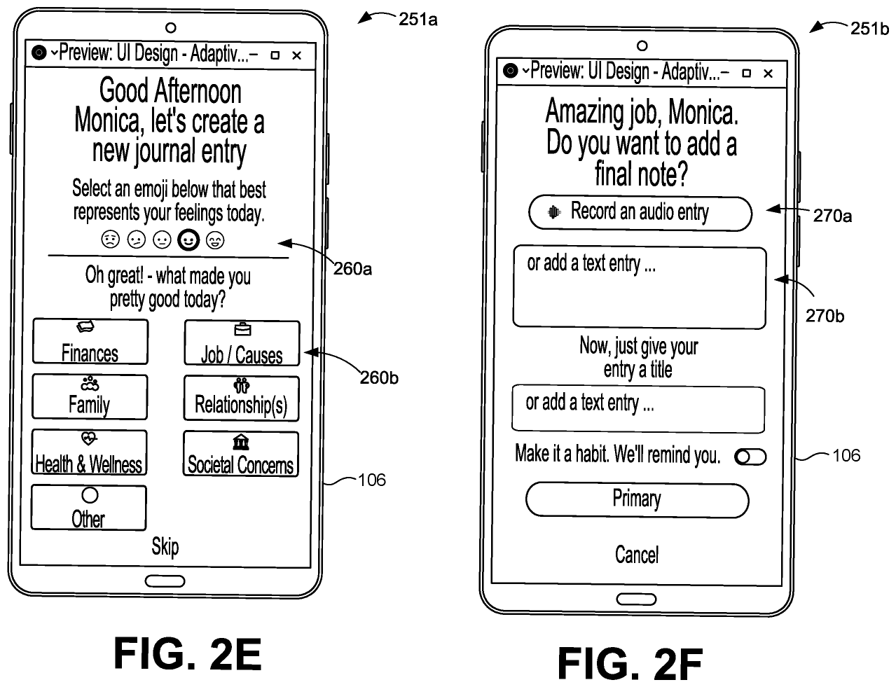

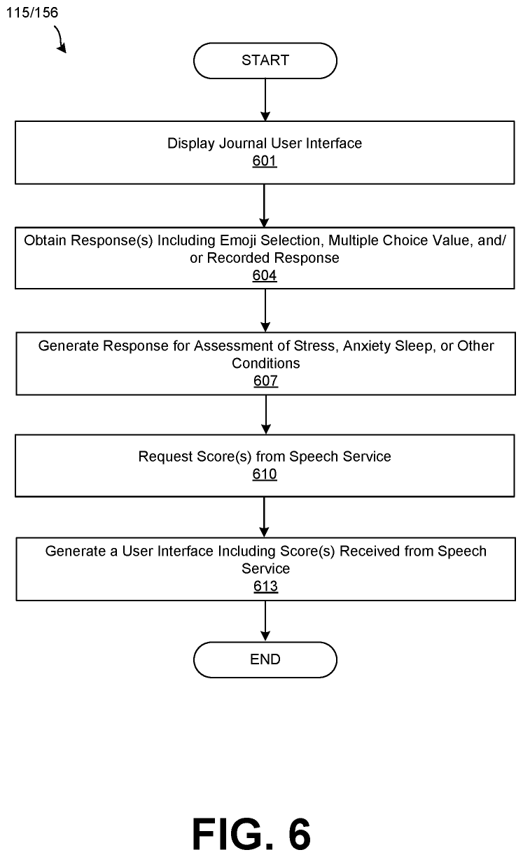

According to one embodiment, the wellness application 115 and/or the client application 156 can be configured to perform journaling functionality, such as described in the journal workflow (see, e.g., FIG. 6). The wellness application 115 can display a journal user interface (see, e.g., FIGS. 2E and 2F) that is configured to receive and store journal entries in different formats. The user can enter text into a text box, in which the entered text can reflect personal thoughts or a status for the user's personal health. The user can also answer multiple choice questions that may reflect a mood or a feeling of the user for the day, such as a selection of a facial emoji. The user can also record an audio sample 128 of the user verbally describing a mood or a feeling, in which the audio sample 128 can be stored as a journal entry for the day the audio sample 128 was recorded.

In some examples, the wellness application 115 can transmit the audio sample 128 for a wellness assessment (e.g. a wellness score 131) to the speech service 118. The speech service 118 can generate the wellness assessment by assessing various biomarkers in the voice recorded in the audio sample 128. After analyzing the biomarkers, the speech service 118 can generate the wellness assessment, which can be transmitted to the wellness application 115. The wellness application 115 can store the wellness assessment in association with a particular day. Accordingly, the wellness assessment can be stored for a particular day in addition to other journal activities (e.g., text input for personal thoughts, answers to the questionnaire on the user's feelings for thatday, etc.).

With reference to FIGS. 2A-2F, shown are various user interfaces (e.g., 201a-d, 251a, and 251b) that the wellness application 115 can transmit to the client application 156 for display on the client device 106. For example, FIGS. 2A-2D illustrate example baseline user interfaces 201a-d for generating baseline data 137 on the initial wellness of the user. Specifically, FIGS. 2A and 2B include baseline user interfaces 201a-d for providing questions/statements 208a and 208b for soliciting an answer response 210a and 210b from the user.

FIG. 2C includes an example of the baseline user interface 201c. The baseline user interface 201c includes an affirmation 220a (e.g., a text script) and a recording component 220b. The recording component 220b can be clicked to initiate the recording of the audio sample 128 while the user reads the affirmation 220a. The affirmation 220a can have a context identifier that uniquely identifies the affirmation 220a from other scripted prompts. In some cases, the affirmation 220a can have a factor in generating the wellness score 131. Also, FIG. 2D includes an example of baseline user interface 201d which illustrates a confirmation that the baseline data has been successfully recorded

FIGS. 2E and 2F illustrate example journal user interfaces 251a, 251b for capturing journal entries in different formats. For example, FIG. 2E illustrates journal user interface 251a that includes an emoji selection 260a (e.g., emotional expression) that is representative of a user's mood. The journal user interface 251a also includes a question for the user to provide an answer in the multiple choice answer selection 260b.

FIG. 2F illustrates the journal user interfaces 251b that includes a recording component 270a for recording the user's voice for a verbal journal entry. The journal user interface 251b also includes a text box 270b for recording a text entry. The journal entries 134 can be stored in association with a particular day in the year, which can be used to track progress toward stress-reduction goals set by the user.

With reference to FIGS. 3A-3D, shown are various user interfaces that the wellness application 115 can transmit to the client application 156 for display on the client device 106. For example, FIG. 3A illustrates a user interface 301a that includes a graph of various wellness scores 131. FIG. 3B illustrates a user interface 301b that includes a reference image 310a for recording an audio sample 128. The user interface 301b includes an audio recording component 310b. The user can click on the audio recording component 310b to initiate a recording of the user verbally describing the reference image 310a.

FIG. 3C is a user interface 301c that illustrates a wellness course (e.g., course content 143) in progress for a user. In this example, the wellness course is on breathing techniques to reduce stress levels. FIG. 3D is a user interface that illustrates a completion of the wellness course (e.g., the wellness course in progress in FIG. 3C).

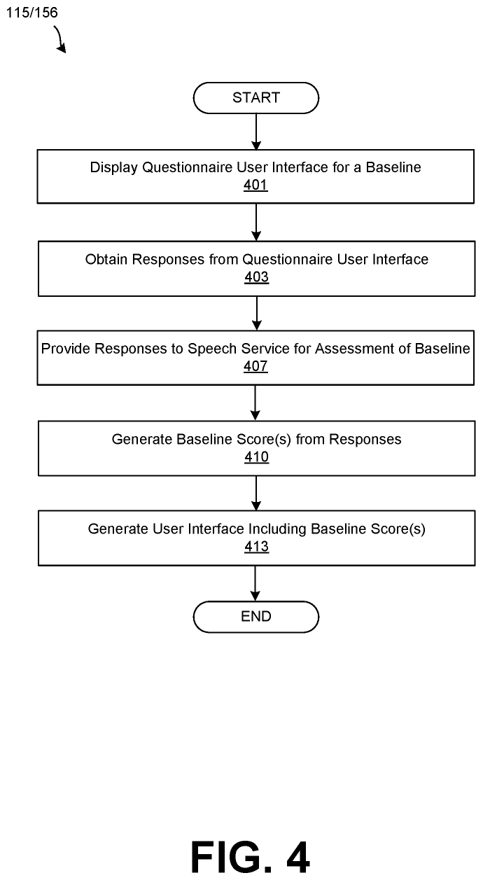

Referring next to FIG. 4, shown is a flowchart that provides one example of baseline workflow functionality implemented as a portion of the wellness application 115 (or the client application 156) according to various embodiments. It is understood that the flowchart of FIG. 4 provides merely an example of the many different types of functional arrangements that may be employed to implement the operation of the portion of the wellness application 115 as described herein. As an alternative, the flowchart of FIG. 4 may be viewed as depicting an example of elements of a method implemented in the computing environment 103 (FIG. 1) according to one or more embodiments.

Beginning with box 401, the wellness application 115 can display a user interface that includes a questionnaire for calculating a wellness baseline. The questionnaire can include one or more questions. The wellness application 115 can store the user responses to the questions in the baseline data 137. The questionnaire can also include a text prompt that instructs the user to verbally recite the text displayed on the user interface 159. In some embodiments, the questionnaire can include an image and provide instructions for a user to verbally describe the image. The audio sample captured from these text prompts or image prompts can be used for analysis.

In box 403, the wellness application 115 can be used to store responses from the questionnaire, text prompts, image prompts, or other suitable prompts. The responses can be stored in the user profile 121, for example in the baseline data 137.

In box 407, the wellness application 115 can provide the responses (e.g., audio samples, response answers) to the speech service 118 for analysis. In some embodiments, the wellness application 115 may transmit a context associated with the responses. For example, the context may include a reference to an image identifier, a question identifier, a text prompt identifier, or other suitable context data. The context can provide the speech service 118 information on the context setting that was used to generate the response. In some non-limiting examples, box 407 may be omitted.

In box 410, the wellness application 115 can generate a baseline score from the responses (e.g., audio samples, response answer, etc.) provided by the client device 106. For example, the speech service 118 can generate a baseline score (e.g., a wellness score 131) based on an analysis of the audio samples 128. The baseline score can reflect a level of stress, anxiety, wellness, and other related health conditions for a user. The baseline score can represent an initial wellness score 131 prior to the user participating in a wellness course, or consuming or applying a wellness product, or any other suitable efforts to improve the wellness of the user. In some embodiments, the client application 156 can generate the baseline score by analyzing the response and the client application 156 and can provide the baseline score to the wellness application 115. Next, in box 413, the wellness application 115 can generate a user interface that displays the baseline score for the user. Then, the wellness application 115 can proceed to the end.

Referring next to FIG. 5, shown is a flowchart that provides one example of course workflow functionality implemented as a portion of the wellness application 115 (or the client application 156) according to various embodiments. It is understood that the flowchart of FIG. 5 provides merely an example of the many different types of functional arrangements that may be employed to implement the operation of the portion of the wellness application 115 (or the client application 156) as described herein. As an alternative, the flowchart of FIG. 5 may be viewed as depicting an example of elements of a method implemented in the computing environment 103 (FIG. 1) according to one or more embodiments.

Beginning with box 501, the wellness application 115 can display on a user interface 159 of the client device 106, the user interface being used to initiate a user to provide a first response. The user interface can include text and a record component for initiating an audio sample. The user interface can include instructions for verbally reciting the text on the user interface 159 while the audio is recorded. For example, the user can first manipulate the record component to initiate the recording. Then, the user can verbally recite the text (e.g. an affirmation 220a) displayed on the user interface. In some embodiments, the user interface may include a reference image (310a) with instructions for the user to verbally describe the image while the audio is recorded. In other examples, the user interface may include questions for the user to answer, such as questions allowing for unstructured text input, multiple choice questions, and other suitable questions.

In box 504, the wellness application 115 can obtain first responses to the prompt user interface. In some examples, the first responses can include an audio sample 128 recorded from the user verbally reciting a portion of text or verbally describing an image on the screen. In other examples, the wellness application 115 can obtain responses to questions displayed on the prompt user interface. For example, the wellness application 115 can record or receive unstructured text answers to a questions, selections of answers to multiple choice questions, and other suitable answers to the questions.

In box 507, the wellness application 115 can generate a first assessment of wellness (e.g., a wellness score 131) for the user based on the first response data provided. In some embodiments, the wellness application 115 can provide the first response data (e.g., audio samples, response answers) to the speech service 118 for analysis. In some embodiments, the wellness application 115 may transmit a context (e.g., an image identifier, a question identifier, a text prompt identifier) associated with the responses. In these embodiments, the wellness application 115 can receive the first assessment of wellness (e.g., a wellness score 131) from the speech service 118. The wellness score 131 can reflect a level of stress, anxiety, or other suitable health related conditions. The first assessment of wellness can reflect the wellness of the user prior to reviewing the wellness content 125 (e.g., course content 143), or consuming or applying a wellness product. In other embodiments, the wellness application 115 and/or the client application 156 can generate the first assessment of wellness based on the first response data.

In box 510, the wellness application 115 can render wellness content 125 on the client device 106. In some examples, the wellness application 115 can stream or render the wellness content 125 that has been selected by the user on the client device 106. In some examples, the wellness application 115 can select wellness content 125 based on the user profile 121 of the user, such as baseline data 137, a first wellness score 131, audio samples 128, and other suitable user profile data. For example, the wellness application 115 can recommend course content 143 that includes guided mediation. The recommendation can also instruct the user to view the course content 143 three times a day.

The wellness application 115 can also recommend one or more products 144 for reducing stress or meeting the wellness goals of the user. For example, based on the wellness score 131, the wellness application 115 can an essential oils products with instructions to take three drops a day. The wellness application 115 may recommend the particular essential oils products based on a pattern of wellness scores 131, based on a particular wellness score 131 reaching an elevated threshold, based on the answers provided to the questionnaire, and other suitable factors. Accordingly, the wellness application 115 can generate product recommendations, recommended dosages for the products 144, recommended duration for the products 144 and other aspects based on one or more wellness score 131.

In box 513, the wellness application 115 can display on a user interface 159 of the client device 106 a user interface to initiate a user to provide a second response after reviewing the wellness content 125. The user interface can include text and a record component for initiating an audio sample. The user interface can include instructions for verbally reciting the text on the user interface 159 while capturing the audio being recorded. In some embodiments, the user interface may include an image with instructions for the user to verbally describe the image while the audio is being recorded. In other examples, the user interface may include questions for the user to answer, such as questions allowing for unstructured text input, multiple choice questions, and other suitable questions.

In box 516, the wellness application 115 can obtain second responses to the user interface. In some examples, the second responses can include an audio sample 128 recorded from the user verbally reciting a portion of text or verbally describing an image on the screen. In other examples, the wellness application 115 can obtain responses to questions displayed on the user interface. For example, the wellness application 115 can record or receive unstructured text answers to questions, selections of answers to multiple choice questions, and other suitable answers to the questions.

In box 519, the wellness application 115 can generate a second assessment of wellness (e.g., wellness score 131) for the second responses and update the user interface to display the second wellness assessment. In response to the second assessment of wellness, the wellness application 115 can display an updated recommendation based on the second assessment of wellness. The wellness application 115 can display a recommendation for a different wellness course, a different dosage of a product 144, or a different product 144. In box 521, the wellness application 115 can display a graph that includes the first assessment of wellness and the second assessment of wellness. Then, the wellness application 115 can proceed to the end.

Referring next to FIG. 6, shown is a flowchart that provides one example of journal workflow functionality implemented as a portion of the wellness application 115 (or the client application 156) according to various embodiments. It is understood that the flowchart of FIG. 6 provides merely an example of the many different types of functional arrangements that may be employed to implement the operation of the portion of the wellness application 115 (or the client application 156) as described herein. As an alternative, the flowchart of FIG. 6 may be viewed as depicting an example of elements of a method implemented in the computing environment 103 (FIG. 1) according to one or more embodiments.

Beginning with box 601, the wellness application 115 can display a journal entry interface on the client device 106. The journal entry interface can be used to receive response data from the user regarding their personal thoughts or health-related issues. The journal entry interface can be used to keep responses from the user over a period of time. In some examples, the journal entries can be stored in in a calendar for a day of the week.

In box 604, the wellness application 115 can obtain the responses from the journal entry interface displayed on the client device 106. The responses may include an emoji selection 260a, a multiple choice answer selection 260b, a recorded audio sample 128, and other suitable journal entry data. The emoji selection 260a can include a selection of a particular facial expression among several that were displayed. The emoji selection 260a can reflect a user's present feeling on a topic or overall feeling for the day. The multiple choice answer selection 260b can be stored in association for a multiple choice question. The recorded audio sample 128 can be captured while a user is verbally reciting a portion of text displayed on the user interface 159 or while the user verbally describing an image displayed on the user interface.

In box 607, the wellness application 115 can generate a response assessment of wellness for the response data provided by the user. In some embodiments, the wellness application 115 can provide the response data (e.g., audio samples, response answers, emoji selections, multiple choice answers, etc.) to the speech service 118 for analysis. In some embodiments, the wellness application 115 may transmit a context (e.g., an image identifier, a question identifier, a text prompt identifier) associated with the responses.

In box 610, the wellness application 115 can request the assessment of wellness (e.g., wellness score 131) from the speech service 118. In some embodiments, box 610 is omitted. In these embodiments, the wellness application 115 and/or the client application can generate the assessment of wellness based on the response data. Next, in box 613, the wellness application 115 can display (for the client device 106) a user interface that includes the assessment of wellness (e.g., the wellness score 131). Then, the wellness application 115 can proceed to the end.

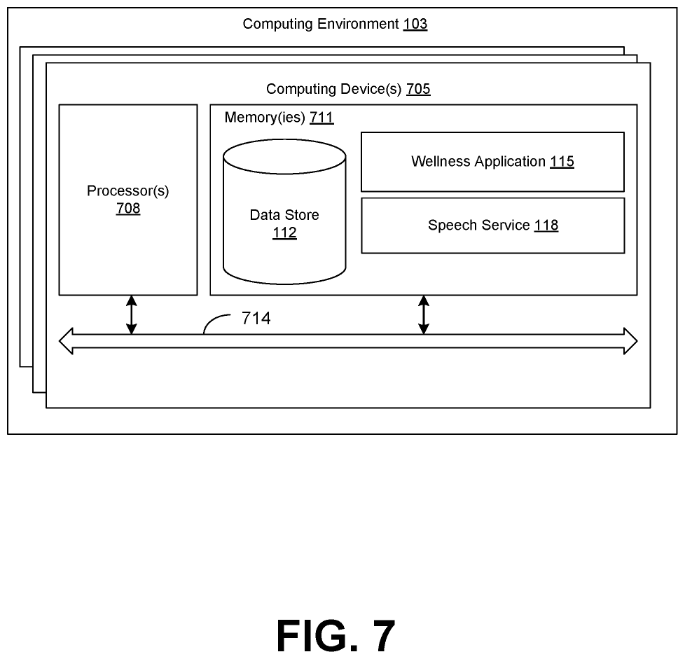

With reference to FIG. 7, shown is a schematic block diagram of the computing environment 103 according to an embodiment of the present disclosure. The computing environment 103 includes one or more computing devices 705. Each computing device 705 includes at least one processor circuit, for example, having a processor 708 and a memory 711, both of which are coupled to a local interface 714. To this end, each computing device 705 may comprise, for example, at least one server computer or like device. The local interface 714 may comprise, for example, a data bus with an accompanying address/control bus or other bus structure as can be appreciated.

Stored in the memory 711 are both data and several components that are executable by the processor 708. In particular, stored in the memory 711 and executable by the processor 708 are the wellness application 115, the speech service 118, and potentially other applications. Also stored in the memory 711 may be a data store 112 and other data. In addition, an operating system may be stored in the memory 711 and executable by the processor 708.

It is understood that there may be other applications that are stored in the memory 711 and are executable by the processor 708 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 711 and are executable by the processor 708. In this respect, the term “executable” means a program file that is in a form that can ultimately be run by the processor 708. Examples of executable programs may be, for example, a compiled program that can be translated into machine code in a format that can be loaded into a random access portion of the memory 711 and run by the processor 708, source code that may be expressed in proper format such as object code that is capable of being loaded into a random access portion of the memory 711 and executed by the processor 708, or source code that may be interpreted by another executable program to generate instructions in a random access portion of the memory 711 to be executed by the processor 708, etc. An executable program may be stored in any portion or component of the memory 711 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 711 is defined herein as including both volatile and nonvolatile memory and data storage components. Volatile components are those that do not retain data values upon loss of power. Nonvolatile components are those that retain data upon a loss of power. Thus, the memory 711 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 708 may represent multiple processors 708 and/or multiple processor cores and the memory 711 may represent multiple memories 711 that operate in parallel processing circuits, respectively. In such a case, the local interface 714 may be an appropriate network that facilitates communication between any two of the multiple processors 708, between any processor 708 and any of the memories 711, or between any two of the memories 711, etc. The local interface 714 may comprise additional systems designed to coordinate this communication, including, for example, performing load balancing. The processor 708 may be of electrical or of some other available construction.

Although the wellness application 115, the speech service 118, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowcharts of FIGS. 4-6 show the functionality and operation of an implementation of portions of the wellness application 115 and the speech service 118. If embodied in software, each block may represent a module, segment, or portion of code that comprises program instructions to implement the specified logical function(s). The program instructions may be embodied in the form of source code that comprises human-readable statements written in a programming language or machine code that comprises numerical instructions recognizable by a suitable execution system such as a processor 708 in a computer system or other system. The machine code may be converted from the source code, etc. If embodied in hardware, each block may represent a circuit or a number of interconnected circuits to implement the specified logical function(s).

Although the flowcharts of FIGS. 4-6 show a specific order of execution, it is understood that the order of execution may differ from that which is depicted. For example, the order of execution of two or more blocks may be scrambled relative to the order shown. Also, two or more blocks shown in succession in FIGS. 4-6 may be executed concurrently or with partial concurrence. Further, in some embodiments, one or more of the blocks shown in FIGS. 4-6 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein, including the wellness application 115 and the speech service 118, that comprises software or code can be embodied in any non-transitory computer-readable medium for use by or in connection with an instruction execution system such as, for example, a processor 708 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the wellness application 115 and the speech service 118, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device 705, or in multiple computing devices in the same computing environment 103. Additionally, it is understood that terms such as “application,” “service,” “system,” “engine,” “module,” and so on may be interchangeable and are not intended to be limiting.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A non-transitory computer-readable medium comprising machine-readable instructions that, when executed by a processor of a computing device, cause the computing device to at least:
display a first wellness prompt on a client device for a first wellness assessment;
receive a first audio sample from the client device, wherein the first audio sample is captured while the first wellness prompt is displayed on the client device;
generate the first wellness assessment based at least in part on the first audio sample;
transmit wellness content to the client device;
display a second wellness prompt on the client device for a second wellness assessment in response to a completion of the wellness content;
receive a second audio sample from the client device, wherein the second audio sample is captured while the second wellness prompt is displayed on the client device;
generate the second wellness assessment based on the second audio sample; and
display the first wellness assessment and the second wellness assessment.

2. The non-transitory computer-readable medium of claim 1, wherein generating the first wellness assessment comprises:
transmitting the first audio sample to a speech service; and
receiving the first wellness assessment from the speech service, wherein the first wellness assessment comprises a score that indicates a level of stress identified in the first audio sample.

3. The non-transitory computer-readable medium of claim 1, wherein the machine-readable instructions, when executed by the processor, cause the computing device to at least:
display a questionnaire user interface that includes at least one question;
receive a user response to the at least one question; and
determine a baseline score based at least in part on the user response.

4. The non-transitory computer-readable medium of claim 1, wherein the first wellness prompt comprises a reference text paragraph or a reference image.

5. The non-transitory computer-readable medium of claim 1, wherein the machine-readable instructions, when executed by the processor, cause the computing device to at least:
display the first wellness assessment and the second wellness assessment on a graph.

6. The non-transitory computer-readable medium of claim 1, wherein at least one of the first wellness assessment and the second wellness assessment are represented by a numerical value that indicates a level of stress or anxiety for the first audio sample or the second audio sample.

7. The non-transitory computer-readable medium of claim 1, wherein displaying the second wellness prompt further comprises:
displaying a reference image and an audio record component; and
initiating a recording of the second audio sample in response to a user manipulation of the audio record component.

8. A system, comprising:
a computing device comprising a processor and a memory; and
machine-readable instructions stored in the memory that, when executed by the processor, cause the computing device to at least:
display a first wellness prompt for a first wellness assessment;
record a first audio sample of a user while the first wellness prompt is displayed;
generate the first wellness assessment based at least in part on the first audio sample;
display wellness content received from a remote computing device;
display a second wellness prompt for a second wellness assessment in response to a completion of the wellness content;
record a second audio sample while the second wellness prompt is displayed;
generate the second wellness assessment based on the second audio sample; and
display the first wellness assessment and the second wellness assessment.

9. The system of claim 8, wherein generating the first wellness assessment comprises:
transmitting the first audio sample to a speech service; and
receiving the first wellness assessment from the speech service, wherein the first wellness assessment comprises a score that indicates a level of stress identified in the first audio sample.

10. The system of claim 8, wherein the machine-readable instructions, when executed by the processor, cause the computing device to at least:
display a questionnaire user interface that includes at least one question;
receive a user response to the at least one question; and
determine a baseline score based at least in part on the user response.

11. The system of claim 8, wherein the first wellness prompt comprises a reference text paragraph or a reference image.

12. The system of claim 8, wherein the machine-readable instructions, when executed by the processor, cause the computing device to at least:
display the first wellness assessment and the second wellness assessment on a graph.

13. The system of claim 8, wherein at least one of the first wellness assessment and the second wellness assessment are represented by a numerical value that indicates a level of stress or anxiety for the first audio sample or the second audio sample.

14. The system of claim 8, wherein displaying the second wellness prompt further comprises:
displaying a reference image and an audio record component; and
initiating a recording of the second audio sample in response to a user manipulation of the audio record component.

15. A computer-implemented method, comprising:
displaying, by a computing device, a first wellness prompt on a client device for a first wellness assessment;
receiving, by the computing device, a first audio sample from the client device, wherein the first audio sample is captured while the first wellness prompt is displayed on the client device;
generating, by the computing device, the first wellness assessment based at least in part on the first audio sample;
transmitting, by the computing device, wellness content to the client device;
displaying, by the computing device, a second wellness prompt on the client device for a second wellness assessment in response to a completion of the wellness content;
receiving, by the computing device, a second audio sample from the client device, wherein the second audio sample is captured while the second wellness prompt is displayed on the client device;
generating, by the computing device, the second wellness assessment based on the second audio sample; and
displaying, by the computing device, the first wellness assessment and the second wellness assessment.

16. The method of claim 15, wherein generating the first wellness assessment comprises:
transmitting the first audio sample to a speech service; and
receiving the first wellness assessment from the speech service.

17. The method of claim 15, wherein the first wellness prompt comprises a reference text paragraph or a reference image.

18. The method of claim 15, further comprising:
displaying, by the computing device, a questionnaire user interface that includes at least one question;
receiving, by the computing device, a user response to the at least one question; and
determining, by the computing device, a baseline score based at least in part on the user response.

19. The method of claim 15, further comprising:
displaying, by the computing device, the first wellness assessment and the second wellness assessment on a graph.

20. The method of claim 15, wherein at least one of the first wellness assessment and the second wellness assessment are represented by a numerical value that indicates a level of stress or anxiety for the first audio sample or the second audio sample.
